(19) United States
(12) Patent Application Publication
(10) Pub. No.: US 2020/0147346 A1
(43) Pub. Date: May 14, 2020

(54) Manual Balloon Articulation Arrays for Catheters and Other Uses

(71) Applicant: Project Moray, Inc., Belmont, CA (US)

(72) Inventors: Douglas Sutton, Pacifica, CA (US); Tim Williams, Palo Alto, CA (US); Keith Phillip Laby, Oakland, CA (US); Mark D. Barrish, Belmont, CA (US)

(73) Assignee: Project Moray, Inc., Belmont, CA (US)

(21) Appl. No.: 16/741,930

(22) Filed: Jan. 14, 2020

Related U.S. Application Data
(63) Continuation of application No. PCT/US2018/042078, filed on Jul. 13, 2018.
(60) Provisional application No. 62/532,654, filed on Jul. 14, 2017.

(51) Int. Cl. A61M 25/01

(52) U.S. Cl. CPC A61M 25/0155

(57) ABSTRACT

Devices, systems, and methods for articulating elongate flexible structures such as catheters optionally include an array of fluid-expandable bodies such as balloons. A user may alter a bend characteristic of the flexible structure by manually moving a handle of a pump. The pump may induce a flow of inflation fluid into a subset of the expandable bodies, and the resulting expansion can change a bend characteristic of the flexible structure. The pump may comprise a threaded syringe pump, one or more balloon that is manually compressed by movement of the handle (so that the balloon acts as a displacement pump), a multi-axis displacement pump (optionally with laterally offset piston-cylinder assemblies coupled to the handle to induce laterally offset bending of the flexible structure), or the like, providing easily-modulated articulation with a low-cost, light-weight, and/or at least partially disposable user interface.

### CROSS REFERENCE TO RELATED APPLICATIONS

The present application is a Continuation of PCT/US2018/042078 filed Jul. 13, 2018; which claims the benefit of U.S. Provisional Appln No. 62/532,654 filed Jul. 14, 2017; the full disclosures which are incorporated herein by reference in their entirety for all purposes.

### FIELD OF THE INVENTION

In general, the present invention provides improved devices, systems, and methods for articulation of elongate flexible bodies such as catheters, borescopes, and the like. In exemplary embodiments, the invention provides manually actuated structures and methods for altering the resting shape (and particularly the axial bending characteristics) of catheters using a fluid-driven articulation balloon array in which at least one subset of balloons in the array is manually inflated by a manual pump.

### BACKGROUND OF THE INVENTION

Diagnosing and treating disease often involve accessing internal tissues of the human body, and open surgery is often the most straightforward approach for gaining access to internal tissues. Although open surgical techniques have been highly successful, they can impose significant trauma to collateral tissues.

To help avoid the trauma associated with open surgery, a number of minimally invasive surgical access and treatment technologies have been developed, including elongate flexible catheter structures that can be advanced along the network of blood vessel lumens extending throughout the body. While generally limiting trauma to the patient, catheter-based endoluminal therapies can be very challenging. Alternative minimally invasive surgical technologies include robotic surgery, and robotic systems for manipulation of flexible catheter bodies from outside the patient have also previously been proposed. Some of those prior robotic catheter systems have met with challenges, in-part because of the difficulties in accurately controlling catheters using pull-wires. While the potential improvements to surgical accuracy make these efforts alluring, the capital equipment costs and overall burden to the healthcare system of these large, specialized systems is a concern.

A new technology for controlling the shape of catheters has recently been proposed which may present significant advantages over pull-wires and other known catheter articulation systems. As more fully explained in US Patent Publication No. US20160279388, entitled “Articulation Systems, Devices, and Methods for Catheters and Other Uses,” published on Sep. 29, 2016 (assigned to the assignee of the subject application and the full disclosure of which is incorporated herein by reference), an articulation balloon array can include subsets of balloons that can be inflated to selectively bend, elongate, or stiffen segments of a catheter. These articulation systems can use pressure from a simple fluid source (such as a pre-pressurized canister) that remains outside a patient to change the shape of a distal portion of a catheter inside the patient via a series of channels in a simple multi-lumen extrusion, providing catheter control beyond what was previously available often without having to resort to a complex robotic gantry, without pull-wires, and even without motors. Hence, these new fluid-driven catheter systems appear to provide significant advantages.

Despite the advantages of the newly proposed fluid-driven catheter system, as with all successes, still further improvements would be desirable. In general, it would be beneficial to provide further improved medical systems, devices, and methods. More specifically, it would be beneficial to facilitate balloon articulation of catheters and other devices without relying on a pre-charged canister or other pressure source, and/or without electronic control of valves, so as to facilitate low-cost manual articulation suitable for third-world markets, catheter bend characteristic changes during manual advancement of a disposable catheter toward the target tissue prior to mounting of the catheter to an automated pressurized fluid supply system, and the like.

### BRIEF SUMMARY OF THE INVENTION

The present invention generally provides improved devices, systems, and methods for articulating elongate flexible structures such as catheters, borescopes, and the like. The structures described herein are particularly well suited for catheter-based therapies, including for cardiovascular procedures such as those in the growing field of structural heart repair, as well as in the broader field of interventional cardiology. Alternative applications may include steerable supports of image acquisition devices such as for trans-esophageal echocardiography (TEE) and other ultrasound techniques, endoscopy, and the like. As a general feature, elongate flexible structures described herein may optionally include an array of fluid-expandable bodies such as balloons. The user will often alter a bend characteristic of the flexible structure by manually moving a handle of a pump. The pump may induce a flow of inflation fluid into a subset of the expandable bodies, and the resulting expansion can change a bend characteristic of the flexible structure. The pump may comprise a threaded syringe pump, one or more balloon that is manually compressed by movement of the handle (so that the balloon acts as a displacement pump), a multi-axis displacement pump (optionally with laterally offset piston-cylinder assemblies coupled to the handle to induce laterally offset bending of the flexible structure), or the like. The systems described herein may provide the advantages of easily-modulated articulation with a low-cost, light-weight, and/or at least partially disposable user interface that is particularly well suited to lower overall healthcare costs.

In a first aspect, the invention provides an articulated catheter system comprising an elongate catheter body having a proximal end and a distal end with an axis therebetween. A balloon array includes a first subset of balloons, the first balloon subset being axially or circumferentially distributed (or both) and offset from the axis. A lumen is in fluid communication with the first balloon subset and extends proximally, and a first manual pump is configured for coupling with the proximal end of the catheter body in fluid communication with the lumen. The first pump has a base and a handle manually movable relative to the base so as to induce a first flow of inflation fluid within the lumen such that the first subset of balloons inflate and induce a first articulation of the catheter body.

Optionally, the balloon array further comprises a second subset of balloons, the second balloon subset being axially distributed and circumferentially offset from the first balloon subset so that the first articulation comprises lateral bending of the catheter body along a first lateral bending axis, and inflation of the second balloon subset induces a second articulation comprising lateral bending of the catheter body along a second lateral bending axis transverse to the first bending axis. The balloon array may further comprise a third subset of balloons, the third balloon subset being axially distributed and axially offset from the first balloon subset so that the first articulation comprises lateral bending of the catheter body along a first axial segment and inflation of the third balloon subset induces a third articulation comprising lateral bending of the axis along a second axial segment axially offset from first segment. In general, the catheter can include a plurality of articulation degrees of freedom (DOFs) with one or more DOF comprising articulation along a first associated axial segment and one or more additional DOF comprising articulation along a second associated axial segment offset from the first associated segment.

Preferably, the first pump comprises a positive displacement pump, and the inflation fluid may comprise an inflation liquid, an inflation gas, or a combination of both. Where additional subsets of balloons are included, they may be inflated by separate manual pumps, by integrated multi-axis manual pump systems, and/or by automated fluid supply systems having powered pumps or other sources of pressurized fluid such as a gas/liquid canister coupled to the balloons by an automated valve system. For example, a second manual pump may be configured for coupling with the proximal end of the catheter body in fluid communication with another lumen, the second pump having a base and a handle manually movable relative to the base so as to induce a second flow of inflation fluid within the other lumen such that the array of balloons articulate the catheter body. The balloon array may include 3 or more associated balloon subsets configured to be coupled to 3 or more associated manual pumps by three or more associated lumens so that the catheter body is configured to articulate with 3 or more degrees of freedom. The balloon array may optionally include 6 or more subsets of balloons so that the catheter body is configured to articulate with 6 or more degrees of freedom. As an optional feature, a first movement of the handle of the first pump relative to the base in a first input orientation induces the first articulation, and a second movement of the handle of the first pump relative to the base in a second orientation induces a fourth articulation.

The manual pumps can take a variety of forms for different uses, and where multiple pumps are included, may be coupled together in a variety of advantageous arrangements. For example, when a plurality of pumps are configured to provide articulation in a plurality of different articulation orientations, the pumps will often have an integrated housing that helps coordinate manual pump handle movement orientations. The handle movement orientations can induce pump flows that articulate the flexible body in corresponding articulation orientations, optionally when the housing is aligned relative to the flexible structure (and/or to an image of the flexible structure provided on a display used for image guided articulation). In one exemplary arrangement, the first pump is configured to be manually reoriented so that the first orientation of the first handle movement corresponds to an image of a first orientation of the first articulation of the catheter body, and so that the second orientation of the second handle movement corresponds to a second orientation of the second handle movement. The first pump may comprise a relatively simple syringe pump, optionally with the pump having threads coupling the handle of the first pump to the base of the first pump so that rotation of the handle relative to the base induces the first flow by driving a piston of the first pump axially within a cylinder of the first pump. Alternatively, the first pump may include a pump balloon in fluid communication with the first subset of balloons, and movement of the handle relative to the base may compress the pump balloon so as to induce a flow on inflation fluid from the pump balloon to the first subset.

In another aspect, the invention provides an articulated catheter for use with a first manual pump having a base. A handle will often be manually movable relative to the base so as to induce a first flow of inflation fluid, and a pump coupler. The catheter comprises an elongate catheter body having a proximal end and a distal end with an axis therebetween. A balloon array includes a first subset of balloons, the first balloon subset being axially distributed and offset from the axis. A lumen is in fluid communication with the first balloon subset and extends proximally, and a catheter coupler is adjacent the proximal end of the catheter body. The catheter coupler is configured for coupling with the pump coupler so as to provide sealed fluid communication between the first pump and the lumen so that the first flow inflates the first subset of balloons and induces a first articulation of the catheter body.

In another aspect, the invention provides a manual pump for use with an articulated catheter. The articulated catheter can include an elongate catheter body, a balloon array including a plurality of subset of balloons, and a plurality of lumens, each lumen in fluid communication with an associated balloon subset, and a catheter coupler. The manual pump may comprise a base, and at least one handle manually movable relative to the base so as to induce a plurality of inflation fluid flows. A pump coupler is configured for coupling with the catheter coupler so as to provide sealed fluid communication between the first pump and the lumens so that the flows inflate the subsets of balloons and each flow induces an associated articulation of the catheter body.

### BRIEF DESCRIPTION OF THE DRAWINGS

FIG. 1 is a simplified perspective view of a medical procedure in which a physician can input commands into a catheter system so that a catheter is articulated using systems and devices described herein.

FIGS. 2A-2C schematically illustrates a catheter having a distal portion with an axial series of articulated segments supporting a prosthetic mitral valve, and show how the segments articulate so as to change the orientation and location of the valve.

FIGS. 3A-3C schematically illustrate input command movements to change the orientation and location of the valve, with the input commands corresponding to the movements of the valve so as to provide intuitive catheter control.

FIG. 4 is a partially see-through perspective view of an exemplary fluid drive manifold system for articulating a balloon array so as to control the shape of a valve delivery catheter or other elongate flexible body.

FIG. 5 is a simplified schematic illustration of components of a helical balloon assembly, showing how an extruded multi-lumen shaft can be assembled to provide fluid to laterally aligned subsets of the balloons.

FIGS. 6A-6C schematically illustrate helical balloon assemblies supported by flat springs and embedded in an elastomeric polymer matrix, and show how selective inflation of subsets of the balloons can elongate and laterally articulate the assemblies.

FIGS. 7 and 8 are cross-sections schematically illustrating a polymer dip coat supporting helical balloon assemblies with the balloons nominally inflated and fully inflated, respectively.

FIGS. 9-11 are cross-sections schematically illustrating a dip-coated helical balloon assembly having a flat spring between axially adjacent balloons in an uninflated state, a nominally inflated state, and a fully inflated state, respectively, with the dip coating comprising a soft elastomeric matrix.

FIG. 12 is a cross-section schematically illustrating yet another alternative dip-coated helical balloon assembly embedded within a relatively soft polymer matrix, with support coils disposed radially inward and outward of the balloon assemblies and dip-coated in a different, relatively hard polymer matrix.

FIGS. 13A-13E schematically illustrate frame systems having axially opposed elongation and contraction balloons for locally elongating and bending a catheter or other elongate flexible body.

FIGS. 14A-14E schematically illustrate frame systems having axially opposed elongation and contraction balloons similar to those of FIGS. 13A-13E, with the frames comprising helical structures.

FIG. 15 is a cross-section schematically illustrating an elongation-contraction frame similar to those of FIGS. 13A-14E, showing a soft elastomeric polymer matrix supporting balloon assemblies within the frames.

FIG. 16 is a drawing of a test manual articulation system in which a plurality of commercially available manual inflation devices are coupled with an associated plurality of subsets of balloons in a flexible segment so as to allow articulation in two lateral degrees of freedom.

FIG. 17 schematically illustrates proximal housings of a modified mitral valve edge-to-edge repair therapy guide catheter and delivery system having manual fluid pumps configured to articulate a flexible catheter system with a plurality of degrees of freedom.

FIGS. 18A and 18B schematically illustrate a multi-segment, multi-DOF (Degree-Of-Freedom) articulated catheter and associated manual pump system.

FIG. 19 schematically illustrates an integrated disposable multi-segment, multi-DOF articulated catheter system having a manual balloon array pump system and an articulation balloon array, the pump balloons including a plurality of balloon subsets in fluid communication with an associated plurality of subsets of the articulation balloon array.

FIG. 20 schematically illustrates a manual pump system having a single gimbal-mounted handle that can be moved in three orthogonal orientations to induce corresponding movement of an articulated catheter.

### DETAILED DESCRIPTION OF THE INVENTION

The present invention generally provides fluid control devices, systems, and methods that are particularly useful for articulating catheters and other elongate flexible structures. The structures described herein will often find applications for diagnosing or treating the disease states of or adjacent to the cardiovascular system, the alimentary tract, the airways, the urogenital system, and/or other lumen systems of a patient body. Other medical tools making use of the articulation systems described herein may be configured for endoscopic procedures, or even for open surgical procedures, such as for supporting, moving and aligning image capture devices, other sensor systems, or energy delivery tools, for tissue retraction or support, for therapeutic tissue remodeling tools, or the like. Alternative elongate flexible bodies that include the articulation technologies described herein may find applications in industrial applications (such as for electronic device assembly or test equipment, for orienting and positioning image acquisition devices, or the like). Still further elongate articulatable devices embodying the techniques described herein may be configured for use in consumer products, for retail applications, for entertainment, or the like, and wherever it is desirable to provide simple articulated assemblies with multiple degrees of freedom without having to resort to complex rigid linkages.

Embodiments provided herein may use balloon-like structures to effect articulation of the elongate catheter or other body. The term “articulation balloon” may be used to refer to a component which expands on inflation with a fluid and is arranged so that on expansion the primary effect is to cause articulation of the elongate body. Note that this use of such a structure is contrasted with a conventional interventional balloon whose primary effect on expansion is to cause substantial radially outward expansion from the outer profile of the overall device, for example to dilate or occlude or anchor in a vessel in which the device is located. Independently, articulated medial structures described herein will often have an articulated distal portion, and an unarticulated proximal portion, which may significantly simplify initial advancement of the structure into a patient using standard catheterization techniques.

The catheter bodies (and many of the other elongate flexible bodies that benefit from the inventions described herein) will often be described herein as having or defining an axis, such that the axis extends along the elongate length of the body. As the bodies are flexible, the local orientation of this axis may vary along the length of the body, and while the axis will often be a central axis defined at or near a center of a cross-section of the body, eccentric axes near an outer surface of the body might also be used. It should be understood, for example, that an elongate structure that extends “along an axis” may have its longest dimension extending in an orientation that has a significant axial component, but the length of that structure need not be precisely parallel to the axis. Similarly, an elongate structure that extends “primarily along the axis” and the like will generally have a length that extends along an orientation that has a greater axial component than components in other orientations orthogonal to the axis. Other orientations may be defined relative to the axis of the body, including orientations that are transvers to the axis (which will encompass orientation that generally extend across the axis, but need not be orthogonal to the axis), orientations that are lateral to the axis (which will encompass orientations that have a significant radial component relative to the axis), orientations that are circumferential relative to the axis (which will encompass orientations that extend around the axis), and the like. The orientations of surfaces may be described herein by reference to the normal of the surface extending away from the structure underlying the surface. As an example, in a simple, solid cylindrical body that has an axis that extends from a proximal end of the body to the distal end of the body, the distal-most end of the body may be described as being distally oriented, the proximal end may be described as being proximally oriented, and the surface between the proximal and distal ends may be described as being radially oriented. As another example, an elongate helical structure extending axially around the above cylindrical body, with the helical structure comprising a wire with a square cross section wrapped around the cylinder at a 20 degree angle, might be described herein as having two opposed axial surfaces (with one being primarily proximally oriented, one being primarily distally oriented). The outermost surface of that wire might be described as being oriented exactly radially outwardly, while the opposed inner surface of the wire might be described as being oriented radially inwardly, and so forth.

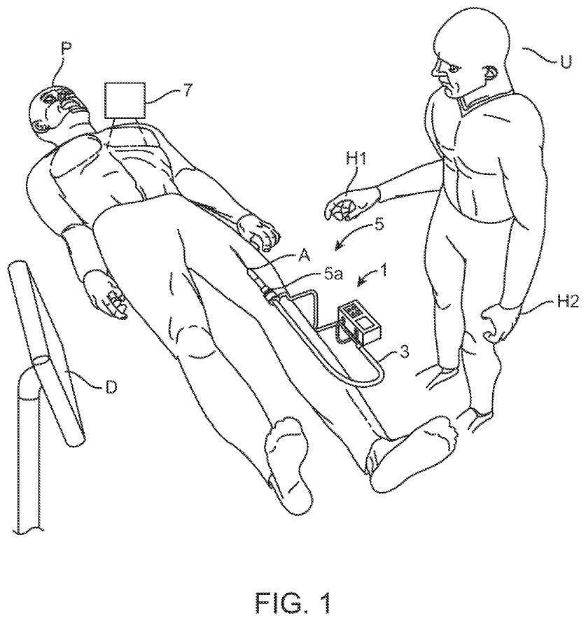

Referring first to FIG. 1, a first exemplary catheter system 1 and method for its use are shown. A physician or other system user U interacts with catheter system 1 so as to perform a therapeutic and/or diagnostic procedure on a patient P, with at least a portion of the procedure being performed by advancing a catheter 3 into a body lumen and aligning an end portion of the catheter with a target tissue of the patient. More specifically, a distal end of catheter 3 is inserted into the patient through an access site A, and is advanced through one of the lumen systems of the body (typically the vasculature network) while user U guides the catheter with reference to images of the catheter and the tissues of the body obtained by a remote imaging system.

Exemplary catheter system 1 will often be introduced into patient P through one of the major blood vessels of the leg, arm, neck, or the like. A variety of known vascular access techniques may also be used, or the system may alternatively be inserted through a body orifice or otherwise enter into any of a number of alternative body lumens. The imaging system will generally include an image capture system 7 for acquiring the remote image data and a display D for presenting images of the internal tissues and adjacent catheter system components. Suitable imaging modalities may include fluoroscopy, computed tomography, magnetic resonance imaging, ultrasonography, combinations of two or more of these, or others.

Catheter 3 may be used by user U in different modes during a single procedure. More specifically, at least a portion of the distal advancement of catheter 3 within the patient may be performed in a manual mode, with system user U manually manipulating the exposed proximal portion of the catheter relative to the patient using hands H1, H2. In addition to such a manual movement mode, catheter system 1 may also have a 3-D automated movement mode using computer controlled articulation of at least a portion of the length of catheter 3 disposed within the body of the patient to change the shape of the catheter portion, often to advance or position the distal end of the catheter. Movement of the distal end of the catheter within the body will often be provided per real-time or near real-time movement commands input by user U. Still further modes of operation of system 1 may also be implemented, including concurrent manual manipulation with automated articulation, for example, with user U manually advancing the proximal shaft through access site A while computer-controlled lateral deflections and/or changes in stiffness over a distal portion of the catheter help the distal end follow a desired path or reduce resistance to the axial movement. Additional details regarding modes of use of catheter 3 can be found in US Patent Publication No. US20160279388, entitled “Articulation Systems, Devices, and Methods for Catheters and Other Uses,” published on Sep. 29, 2016, assigned to the assignee of the subject application, the full disclosure of which is incorporated herein by reference.

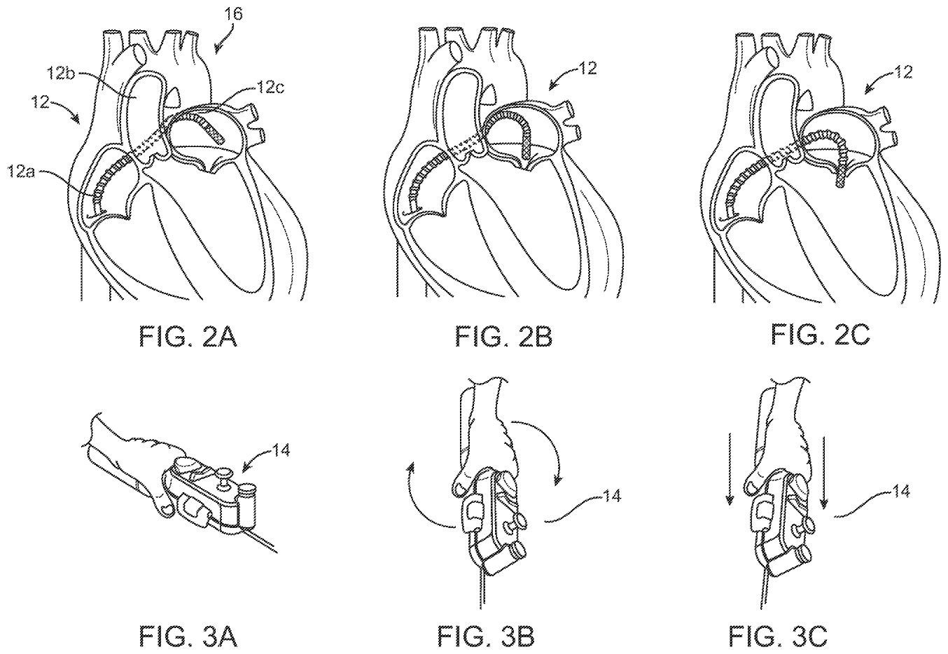

Referring now to FIGS. 2A-3C, devices and methods are shown for controlling movement of the distal end of a multi-segment articulated catheter 12 using a movement command input device 14 in a catheter system similar system 1 (described above). Multi-segment catheter 12 is shown in FIG. 2A extending within a heart 16, and more specifically with a distal portion of the catheter extending up to the heart via the inferior vena cava, with a first, proximal articulatable segment 12a bending within a right atrium of the heart toward a trans-septal access site. A second, intermediate articulatable segment 12b traverses the septum, and a third, distal articulatable segment 12c has some bend inside the left atrium of the heart 16. A tool, such as a prosthetic mitral valve, is supported by the distal segment 12c, and the tool is not in the desired position or orientation for use in the image of FIG. 2A. As shown in FIG. 3A, input device 14 is held by the hand of the user in an orientation that, very roughly, corresponds to the orientation of the tool (typically as the tool is displayed to the user in the display of the image capture system, as described above).

Referring to FIGS. 2A, 2B, 3A, and 3B, to change an orientation of the tool within the heart the user may change an orientation of input device 14, with the schematic illustration showing the input command movement comprising a movement of the housing of the overall input device. The change in orientation can be sensed by sensors supported by the input housing (with the sensors optionally comprising orientation or pose sensors similar to those of smart phones, tablets, game controllers, or the like). In response to this input, the proximal, intermediate, and distal segments 12a, 12b, and 12c of catheter 12 may all change shape so as to produce the commanded change in orientation of the tool. The changes in shapes of the segments will be calculated by a robotic processor of the catheter system, and the user may monitor the implementation of the commanded movement via the image system display. Similarly, as can be understood with reference to FIGS. 2B, 2C, 3B, and 3C, to change a position of the tool within the heart the user may translate input device 14. The commanded change in position can again be sensed and used to calculate changes in shape to the proximal, intermediate, and distal segments 12a, 12b, and 12c of catheter 12 so as to produce the commanded translation of the tool. Note that even a simple change in position or orientation (or both) will often result in changes to shape in multiple articulated segments of the catheter, particularly when the input movement command (and the resulting tool output movement) occur in three dimensional space within the patient.

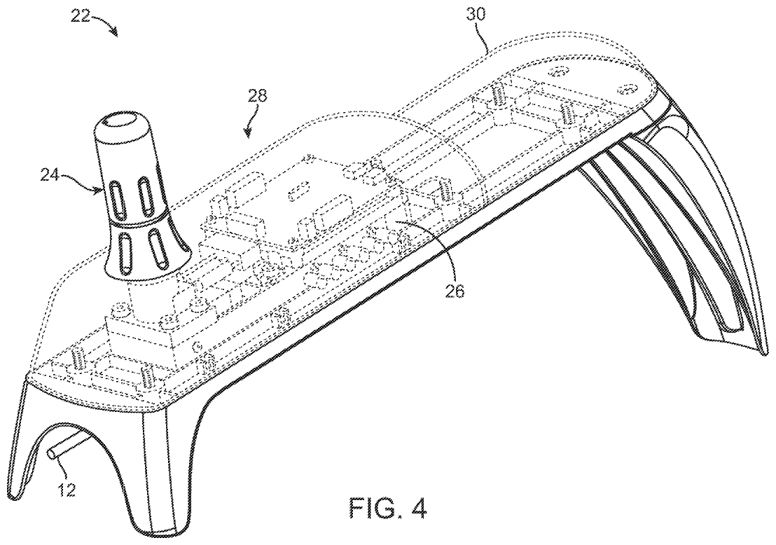

Referring to FIG. 4, an exemplary articulated catheter drive system 22 includes a pressurized fluid source 24 coupled to catheter 12 by a manifold 26. The fluid source preferably comprises a receptacle for and associated disposable canister containing a liquid/gas mixture, such as a commercially available nitrous oxide (N20) canister. Manifold 26 may have a series of valves and pressure sensors, and may optionally include a reservoir of a biocompatible fluid such as saline that can be maintained at pressure by gas from the canister. The valves and reservoir pressure may be controlled by a processor 28, and a housing 30 of drive system 22 may support a user interface configured for inputting of movement commands for the distal portion of the catheter, as more fully explained in co-pending U.S. patent application Ser. No. 15/369,606 (now U.S. Pat. No. 10,525,233), entitled “INPUT AND ARTICULATION SYSTEM FOR CATHETERS AND OTHER USES,” filed on Dec. 5, 2016 (the full disclosure of which is incorporated herein by reference).

Regarding processor 28 and the other data processing components of drive system 22, it should be understood that a variety of data processing architectures may be employed. The processor, pressure or position sensors, and user interface will, taken together, typically include both data processing hardware and software, with the hardware including an input (such as a joystick or the like that is movable relative to housing 30 or some other input base in at least 2 dimensions), an output (such as a sound generator, indicator lights, and/or an image display, and one or more processor board. These components are included in a processor system capable of performing the rigid-body transformations, kinematic analysis, and matrix processing functionality associated with generating the valve commands, along with the appropriate connectors, conductors, wireless telemetry, and the like. The processing capabilities may be centralized in a single processor board, or may be distributed among the various components so that smaller volumes of higher-level data can be transmitted. The processor(s) will often include one or more memory or storage media, and the functionality used to perform the methods described herein will often include software or firmware embodied therein. The software will typically comprise machine-readable programming code or instructions embodied in non-volatile media, and may be arranged in a wide variety of alternative code architectures, varying from a single monolithic code running on a single processor to a large number of specialized subroutines being run in parallel on a number of separate processor sub-units.

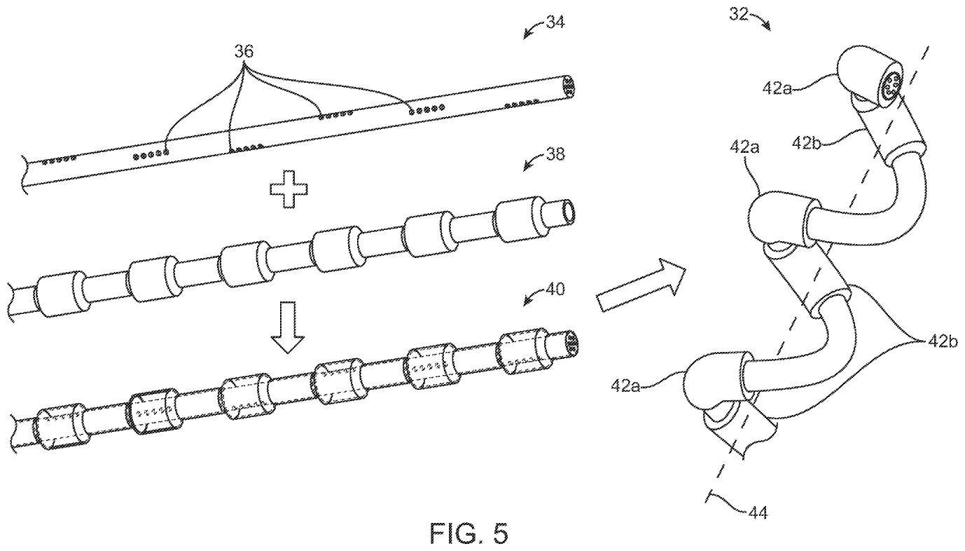

Referring now to FIG. 5, the components of, and fabrication method for production of, an exemplary balloon array assembly, sometimes referred to herein as a balloon string 32, can be understood. A multi-lumen shaft 34 will typically have between 3 and 18 lumens. The shaft can be formed by extrusion with a polymer such as a nylon, a polyurethane, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like. A series of ports 36 are formed between the outer surface of shaft 36 and the lumens, and a continuous balloon tube 38 is slid over the shaft and ports, with the ports being disposed in large profile regions of the tube and the tube being sealed over the shaft along the small profile regions of the tube between ports to form a series of balloons. The balloon tube may be formed using any compliant, non-compliant, or semi-compliant balloon material such as a latex, a silicone, a nylon elastomer, a polyurethane, a nylon, a thermoplastic such as a Pebax™ thermoplastic or a polyether ether ketone (PEEK) thermoplastic, a polyethylene terephthalate (PET) polymer, a polytetrafluoroethylene (PTFE) polymer, or the like, with the large-profile regions preferably being blown sequentially or simultaneously to provide desired hoop strength. The shaft balloon assembly 40 can be coiled to a helical balloon array of balloon string 32, with one subset of balloons 42a being aligned along one side of the helical axis 44, another subset of balloons 44b (typically offset from the first set by 120 degrees) aligned along another side, and a third set (shown schematically as deflated) along a third side. Alternative embodiments may have four subsets of balloons arranged in quadrature about axis 44, with 90 degrees between adjacent sets of balloons.

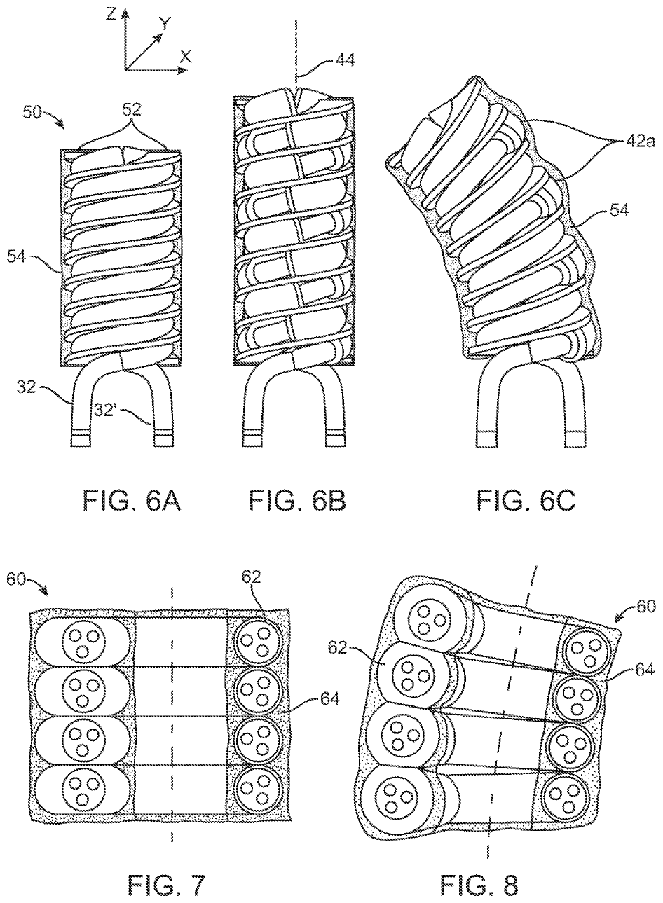

Referring now to FIGS. 6A, 6B, and 6C, an articulated segment assembly 50 has a plurality of helical balloon strings 32, 32′ arranged in a double helix configuration. A pair of flat springs 52 are interleaved between the balloon strings and can help axially compress the assembly and urge deflation of the balloons. As can be understood by a comparison of FIGS. 6A and 6B, inflation of subsets of the balloons surrounding the axis of segment 50 can induce axial elongation of the segment. As can be understood with reference to FIGS. 6A and 6C, selective inflation of a balloon subset 42a offset from the segment axis 44 along a common lateral bending orientation X induces lateral bending of the axis 44 away from the inflated balloons. Variable inflation of three or four subsets of balloons (via three or four channels of a single multi-lumen shaft, for example) can provide control over the articulation of segment 50 in three degrees of freedom, i.e., lateral bending in the +/−X orientation and the +/−Y orientation, and elongation in the +Z orientation. As noted above, each multilumen shaft of the balloon strings 32, 32′ may have more than three channels (with the exemplary shafts having 6 lumens), so that the total balloon array may include a series of independently articulatable segments (each having 3 or 4 dedicated lumens of one of the multi-lumen shafts, for example).

Referring still to FIGS. 6A, 6B, and 6C, articulated segment 50 includes a polymer matrix 54, with some or all of the outer surface of balloon strings 32, 32′ and flat springs 52 that are included in the segment being covered by the matrix. Matrix 54 may comprise, for example, a relatively soft elastomer to accommodate inflation of the balloons and associated articulation of the segment, with the matrix optionally helping to urge the balloons toward an at least nominally deflated state, and to urge the segment toward a straight, minimal length configuration. Advantageously, matrix 54 can maintain overall alignment of the balloon array and springs within the segment despite segment articulation and bending of the segment by environmental forces.

Segment 50 may be assembled by, for example, winding springs 52 together over a mandrel and restraining the springs with open channels between the axially opposed spring surfaces. Balloon strings 32, 32′ can be wrapped over the mandrel in the open channels. The balloons may be fully inflated, partially inflated, nominally inflated (sufficiently inflated to promote engagement of the balloon wall against the opposed surfaces of the adjacent springs without driving the springs significantly wider apart than the diameter of the balloon string between balloons), deflated, or deflated with a vacuum applied to locally flatten and maintain 2 or 4 opposed outwardly protruding pleats or wings of the balloons. The balloons may be pre-folded, gently pre-formed at a moderate temperature to bias the balloons toward a desired fold pattern, or unfolded and constrained by adjacent components of the segment (such as the opposed surfaces of the springs and/or other adjacent structures) urge the balloons toward a consistent deflated shape. When in the desired configuration, the mandrel, balloon strings, and springs can then be dip-coated in a pre-cursor liquid material of polymer matrix 54, with repeated dip-coatings optionally being performed to embed the balloon strings and springs in the matrix material and provide a desired outer coating thickness. Alternatively, matrix 54 can be over-molded onto, sprayed or poured over the balloon strings and springs, or the like. The liquid material can be evened by rotating the coated assembly, by passing the assembly through an aperture, by manually troweling matrix material over the assembly, or the like. Curing of the matrix may be provided by heating (optionally while rotating about the axis), by application of light, by inclusion of a cross-linking agent in the matrix, or the like. The polymer matrix may remain quite soft in some embodiments, optionally having a Shore A durometer hardness of 2-30, typically being 3-25, and optionally being almost gel-like. Other polymer matrix materials may be somewhat harder (and optionally being used in somewhat thinner layers), having Shore A hardness durometers in a range from about 20 to 95, optionally being from about 30 to about 60. Suitable matrix materials comprise elastomeric polyurethane polymers, silicone polymers, latex polymers, polyisoprene polymers, nitrile polymers, plastisol polymers, or the like. Regardless, once the polymer matrix is in the desired configuration, the balloon strings, springs, and matrix can be removed from the mandrel. Optionally, flexible inner and/or outer sheath layers may be added.

Referring now to FIGS. 7 and 8, a simple articulated segment 60 includes a single balloon string 62 supported by a polymer matrix 64 in which the balloon string is embedded. A multilumen shaft of balloon string 62 includes 3 lumens, and the balloons of the balloon string are shown in a nominally inflated state in FIG. 7, so that the opposed major surfaces of most of the balloons of each subset are disposed between and adjacent balloons of that subset on adjacent loops, such that pressure within the subset of balloons causes the balloons to push away from each other (see FIG. 8). Optionally, the balloons of the subset may directly engage each other across much or all of the balloon/balloon force transmission interface, particularly when the balloons are dip-coated when in the nominally inflated state. Alternatively, a layer of matrix 64 may be disposed between some portion or all of the adjacent force-transmission balloon wall surfaces of the subset, for example, if the balloon strings are dip-coated in a deflated state. As can be understood with reference to FIG. 8, inflation of one or more subsets of the balloons may separate adjacent loops of the balloon string between balloons, along the tapering balloon ends, and the like. Elastic elongation of matrix 64 may accommodate some or all of this separation, or the matrix may at least locally detach from the outer surface of the balloon string to accommodate the movement. In some embodiments, localized fracturing of the polymer matrix in areas of high elongation may help to accommodate the pressure-induced articulation, with the overall bulk and shape of the relatively soft matrix material still helping to keep the balloons of the helical balloon array in the desired alignment.

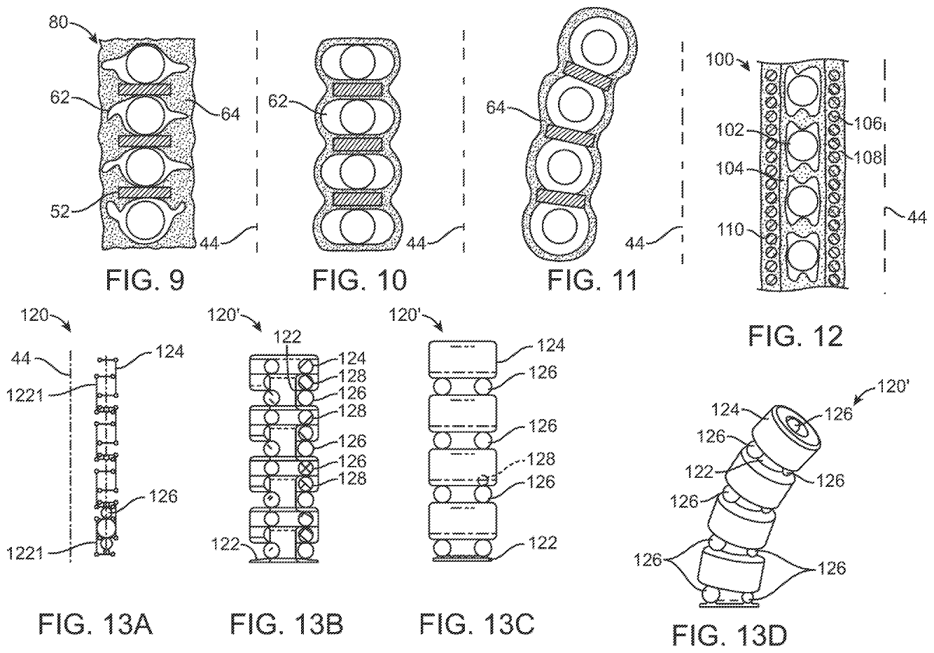

Referring now to FIGS. 9-11, an alternative segment 80 has a single balloon string 62 interleaved with a flat spring 52, and both the balloon string and spring are coated by an elastomeric polymer matrix 64. Shape setting of the balloons may be optionally be omitted, as axial compression of spring 52 can help induce at least rough organization of deflated balloons 62 (as shown in FIG. 9). Local inclusion of some matrix material 64 between the balloon walls and adjacent spring surface (see FIG. 10) may not significantly impact overall force transmission and articulation, particularly where the balloons are generally oriented with major surfaces in apposition, as the pressure force can be transmitted axially through the soft matrix material. Alternatively, the balloons may be nominally inflated during application of the matrix material, as noted above, providing a more direct balloon wall/spring interface (see FIG. 11). As with the other embodiments of segments described herein, flexible (and often axially resilient) radially inner and/or outer sheaths may be included, with the sheaths optionally comprising a coil or braid to provide radial strength and accommodate bending and local axial elongation, such inner and/or outer sheaths often providing a barrier to inhibit release of inflation fluid from the segment should a balloon string leak.

Referring now to FIG. 12, an exemplary segment 100 was fabricated with an intermediate sub-assembly including balloon string 102 embedded in an intermediate matrix 104. An inner sheath is formed radially inward of (and optionally prior to the assembly of) the intermediate sub-assembly by embedding an inner spring 106 within an inner matrix 108. An outer sheath is formed radially outward of (and optionally after assembly of) the intermediate assembly, with the outer sheath including an outer spring 110 and an outer matrix. Note that as in this embodiment, it will often be beneficial for any inner or outer spring to be counterwound relative to the balloon string. First, when the loops of the springs cross the balloons it may help inhibit radial protrusion of the balloons through the coils. Second, it may help to counteract rotational unwinding of the balloon coil structure with balloon inflation, and thereby inhibit non-planar articulation of the segment form inflation of a single balloon subset. Alternative embodiments may benefit from harder matrix materials encompassing the inner or outer springs (or both), from replacing the inner or outer springs (or both) with a braid or eliminating the springs altogether, or the like.

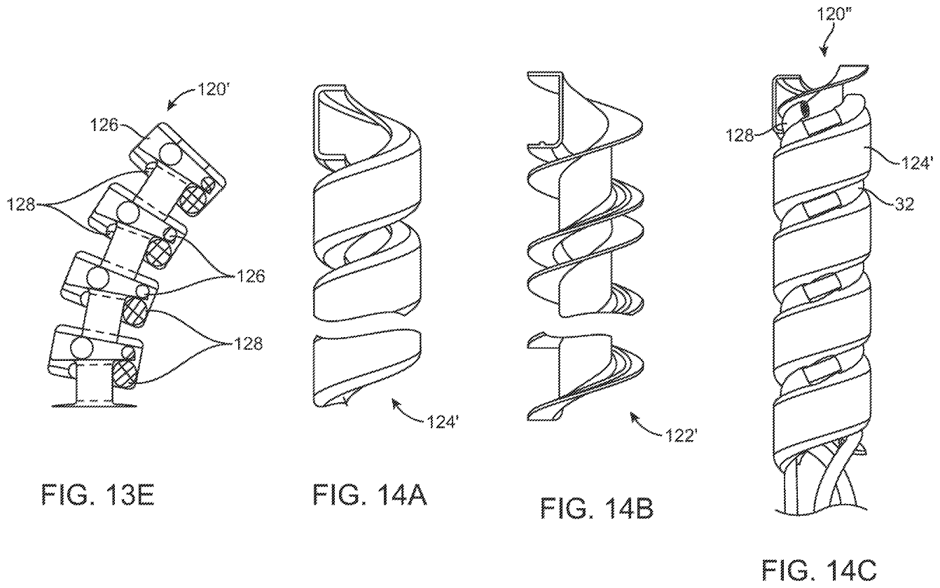

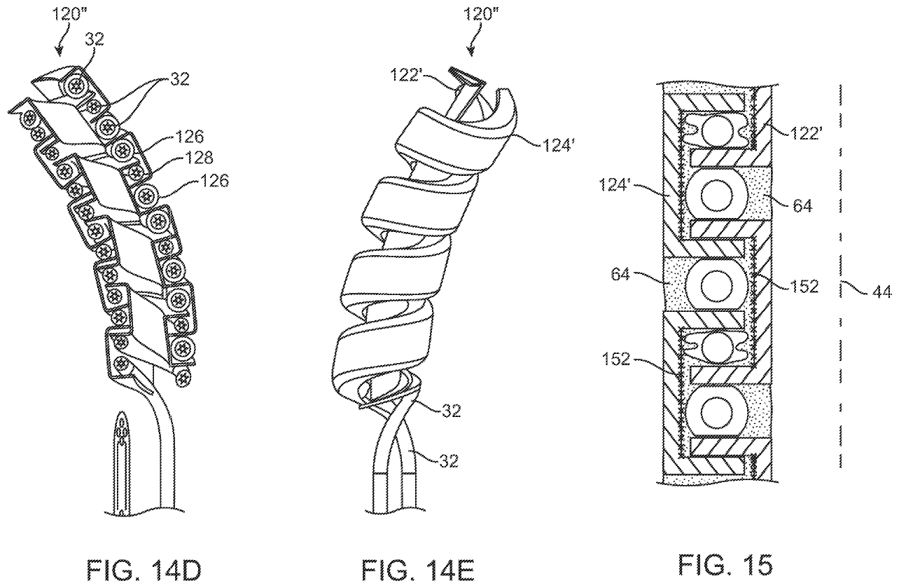

Referring now to FIGS. 13A-14E, alternative segment structures include opposed balloons disposed within channels of segment frames or skeletons to locally axially elongate or contract the frame, thereby laterally bending the frame or changing the axial length of the frame. Referring first to FIG. 13A, a schematically illustrated frame structure 120 includes an axially interleaved set of frame members, with an inner frame 122 having a radially outwardly open channel, and an outer frame 124 having a radially inwardly open channel. The channels are both axially bordered by flanges, and radially bordered (at an inner or outer border of the channel) by a wall extending along the axis. A flange of the inner frame extends into the channel of the outer frame, and a flange of the outer frame extends into the channel of the inner frame. Axial extension balloons 126 can be placed between adjacent flanges of two inner frames or between flanges of two adjacent outer frames; axial retraction balloons 128 can be placed between a flange of an inner frame and an adjacent flange of an outer frame. As more fully explained in US Patent Publication No. US20160279388, entitled “Articulation Systems, Devices, and Methods for Catheters and Other Uses,” published on Sep. 29, 2016 (assigned to the assignee of the subject application and the full disclosure of which is incorporated herein by reference), inflation of a subset of extension balloons 126 along one side of the frame locally extends the axial length of the frame and can bend the frame away from the balloons of the subset. A subset of retraction balloons 128 is mounted in opposition to that local extension, so that inflation of those retraction balloons (with concurrent deflation of the extension balloons) may move the flanges between the balloons in the opposed direction, locally decreasing the length of the frame and bending the axis of the frame toward the inflating retraction balloons. As can be understood with reference to FIGS. 13B-13E, annular frame segments 120′ may have an axially series of ring-shaped inner and outer frames defining the flanges and channels. As shown in FIGS. 14A-14E, helical versions of the frame system may have helical inner and outer frame members 122′, 124′, with extension balloons 126 and retraction balloons 128 being disposed on multiple helical balloon strings extending along the helical channels.

Referring now to FIG. 15, embedding the balloons within the helical frames 122′, 124′ or ring frames described herein within polymer matrix 64 may help maintain alignment of the subsets of balloons despite frame articulation. Articulation performance may be enhanced by the use of soft matrices (with Shore A durometers of 2 to 15), and by inhibiting adhesion at the frame/matrix interface 152 between the axial wall of the frames and the matrix in the channels. Preferably, a slippery interface 152 is provided by a low-friction surface in the channels of the frames between flanges, such as by coating the axial walls with a mold release agent, a PTFE polymer coating or flange material, or the like.

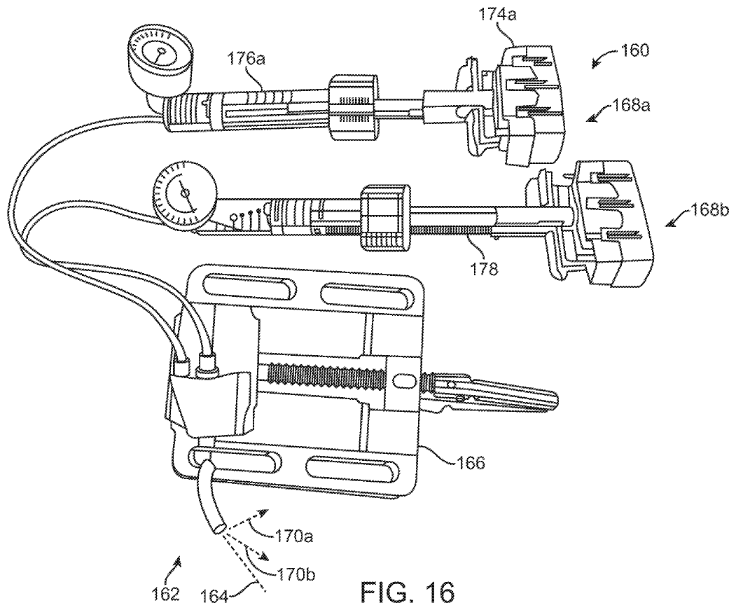

Referring now to FIGS. 16 and 16A, in a test system 160, a multiple degree-of-freedom articulated segment 162 has an axis 164 and is supported by a base, the base here being a vise 166. First and second manual pumps 168a, 168b are each coupled to associated subsets of balloons in a balloon array with the subsets each being aligned and offset from axis 164 that inflation of a first balloon subset induces lateral bending of segment 162 in the direction of lateral bending axis 170a; inflation of a second subset similarly induces bending toward lateral bending axis 170b. Manual articulation of a handle of a pump relative to a base of the pump (such as handle 174a of pump 168a relative to base 176a) induces a flow of inflation fluid between the pump and the associated balloon subset, thereby articulating the segment.

In test system 160, pumps 168a, 168b comprise commercially available inflation devices sometimes referred to as endoflators or insuflators, and often used to inflate medical balloons for percutaneous coronary interventions such as angioplasty, stenting, and the like. The exemplary test system uses BIG60™ inflation devices commercially available from Merit Medical of Utah. The handle may be twisted about an axis so that corresponding threads 178 of the handle and base of the pump move a syringe piston axially within a corresponding cylinder, or the handle may be moved axially (optionally by squeezing or otherwise actuating a thread detachment latch). Axial movement of the handle is particularly well-suited for priming and low-pressure articulations, while threaded twisting of the handle may be well suited for higher pressure and/or finer movements. A fluid pressure indicator for each pump helps to provide feedback to the system operator regarding balloon pressures on the subset of balloons in fluid communication with that pump. A variety of alternative manual pumps can be used, with preferred manual pumps configured to provide pressures of up to at least 10 atm, typically of at least 15 atm, most often at least 20 atm, in many cases at least 25 atm, and in some cases at least 30 atm or more. Volumes of fluid manually pumped to vary inflation of balloon subsets during articulation can be 30 cc or more when gas is used for balloon inflation, often being 50 cc or more. Compression of these relatively large volumes of gas may optionally be performed manually, by a powered compression pump system, or using a hybrid manual/powered pump system. For example, when large pressure changes are desired a hybrid manual/powered pump may allow the user to energize a motor that rotates a manual pump handle. Similarly, a syringe pump system may include a ball screw component to drive a piston in a linear fashion using a rotary motor. Hence, manual pump systems need not be manual-only pump systems that are always manually actuated. Liquid inflation fluids such as saline or the like may facilitate manual pumping for articulation by limiting displacement pump actuation, with exemplary inflation fluid flows during articulation of a single cardiac catheter articulation balloon subset being less than 20 cc, often being less than 10 cc, and optionally being less than 5 cc. Larger displacement pump volumes may optionally be provided for system priming and the like, or priming may be facilitated by another source of pressurized inflation fluid.

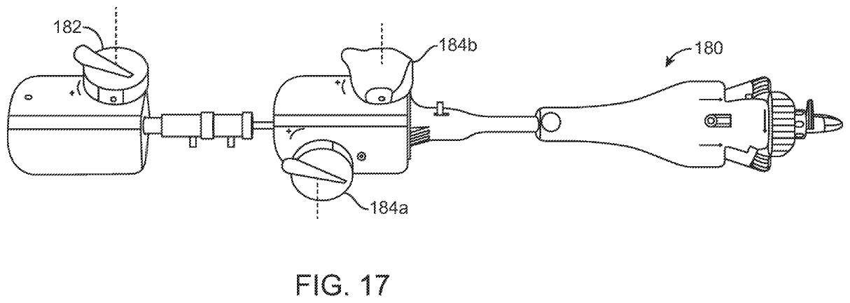

Referring now to FIGS. 17 and 17A, an exemplary manual valve therapy catheter articulation pump system 180 is shown schematically, with manual pumps 182 (mounted to a proximal housing of a guide catheter), 184a, and 184b (mounted to a proximal housing of an implant delivery catheter extending through the guide catheter). The pump handle arrangement can thus mimic the pull-wire handles of pull-wire articulated catheter delivery systems such as that developed by E-Valve, now commercially available from Abbott as the MitraClip™ system. Each pump may optionally include a threaded interface between a piston and a cylinder, so that rotation of the handle induces axial movement of the piston to direct flow to or from an associated balloon subset. Additional degrees of freedom of the delivery system may be provided by supporting the proximal housings so as to accommodate axial rotation and/or axial movement in a nested catheter arrangement. In some embodiments, pull-wire articulation in one or more degree of freedom (such as lateral bending of an outer guide catheter) may be combined with fluid-driven manual articulation in one or more degree of freedom.

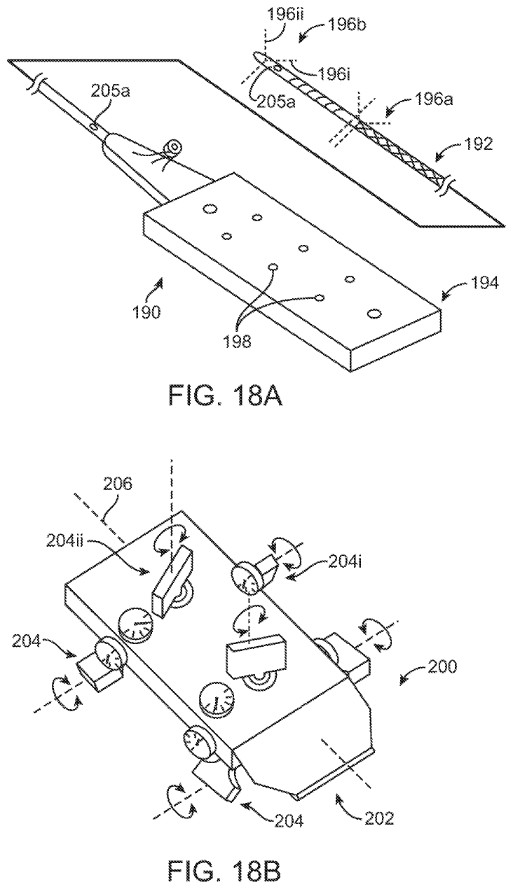

Referring now to FIGS. 18A and 18B, a disposable catheter 190 includes a flexible catheter body 192 extending distally from a connector 194. A balloon array is included in first and second independently articulatable segments 196a, 196b, with the balloon arrays including three subsets aligned along associated lateral bending orientations as described above. Connector 194 includes an array of ports 198, with each port being in fluid communication with a lumen extending along the catheter body to the balloons of an associated substrate. A manual pump assembly 200 has an interface surface 202 that engages connector 194 so as to provide sealed communication between each port 198 and an associated manual pump 204. In the schematic illustration of FIG. 18B, each pump is shown with an optional associated pressure gauge and with a handle that can be turned relative to a housing of the pump assembly to induce fluid flow to or from the associated balloon subgroup via a threaded engagement between a piston (coupled to the handle) and an associated cylinder (coupled to the housing), as described above. A wide variety of alternative handle/housing mechanical couplings might be employed.

The pumps 204 of pump assembly 200 are arranged to facilitate control over the multiple degrees of freedom of the segments. More specifically, pumps 204 used to articulate proximal segment 196a are axially offset along an axis 206 of the housing from the pumps driving distal segment 196b, and the housing is sized and shaped so as to facilitate moving the housing with one hand into axial alignment with the articulated catheter segments (or an image thereof). Additionally, the pumps driving each segment are arranged around the axis of the housing so as to circumferentially correspond to the lateral bending axes of the associated subsets of balloons. Hence, articulation of a first pump 204i on a first side of housing 202 alters bending of segment 196b in a first lateral bending orientation 196i; and articulation of a second pump 204ii on a second side of housing 202 (offset from the first pump in a circumferential direction) alters bending of segment 196b in a second lateral bending orientation 196ii (also offset from the first bending orientation in the same circumferential orientation). A third (and if present, a fourth) pump and bending orientation for the same segment may be further offset in the same circumferential orientation, and pumps for different segments may be axially aligned. Use of these corresponding pump positions and bending orientations may be facilitated by rotational alignment provided by the catheter connector and housing interface (which can be configured to provide and maintain alignment about the axes of the pump assembly and catheter), by a rotationally stiff catheter structure, by a rotational marking on the housing 205b and a corresponding rotational radiopaque marker 205a of the articulatable portion of the catheter, and the like.

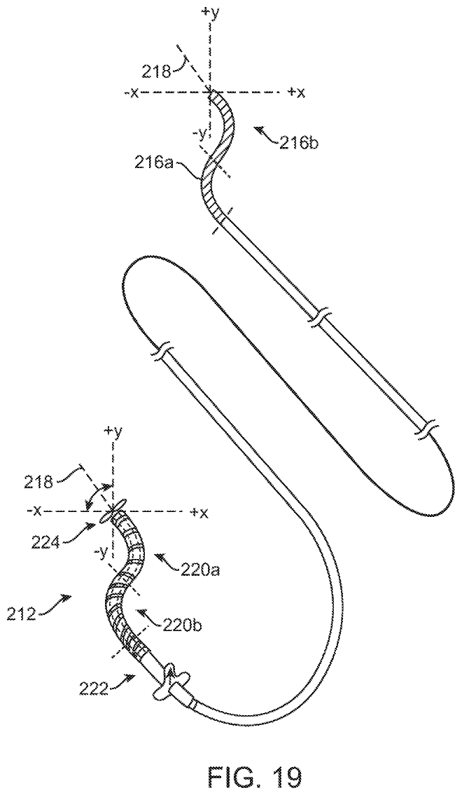

Referring now to FIG. 19, a still further alternative articulated system 210 having a balloon pump array drive system 212 is schematically illustrated. In this embodiment, a flexible catheter 214 extends distally from balloon pump drive system to an articulated portion having first and second segments 216a, 216b. As described above, each segment 216 includes a balloon array with a plurality of balloon subsets, the subsets here arranged in quadrature about an axis 218 of the catheter (so that balloons of the segment along a +X lateral bending orientation are in fluid communication along an associated lumen, balloons along a −X orientation along a different lumen, +Y along a third lumen, and −Y along a fourth lumen). For two segments, 8 lumens may be provided, and the balloon array may be arranged along one or more multi-lumen helical assembly as described above.

To provide and control inflation fluid to articulated segments 216a, 216b, balloon pump drive system 212 also includes a balloon array with subsets arranged in quadrature for each segment. Hence in a first segment 220a, balloon subsets are arranged in alignment with the +X, −X, +Y, and −Y lateral bending axes about catheter axis 218. A second segment 220b has a similar balloon array in quadrature. A rigid housing portion 222 extends between the pump balloon arrays and the flexible catheter body (for supporting the drive system with one hand during use), and a resilient handle structure (such as an outer metallic coil or the like) helps support the pump balloon arrays and urges the segments toward a constant curvature configuration. Note that the proximal segment 220a of the balloon pump drive system is here schematically shown aligned with the distal segment 216b of the articulated portion, as end-end alignment may be easier. Moreover, the system may benefit from both opposed axial segment coupling (for example, with the proximal-most pump balloon segment coupled to the distal-most catheter articulation segment, and the distal-most pump balloon segment coupled to the proximal-most catheter articulation segment) and laterally opposed balloon subset coupling (for example, with each subset of a particular segment of the pump balloon array being in fluid communication with a catheter balloon subset of an associated catheter segment that is 180 degrees opposed about the catheter axis) to provide corresponding lateral articulations that are intuitive to the user. Axial articulation of the pump and catheter balloon subsets will tend to be in opposed axial directions (for example, when the overall pump balloon array elongates, the catheter balloon array may shorten). A visual lateral orientation indicator is shown affixed to the rigid portion 222, and a corresponding radiopaque marker adjacent the articulated portion of the catheter can help provide rotational alignment about the catheter axis.

To induce articulation of the catheter segments corresponding to the articulation of the balloon drive system handle, a lumen may provide fluid communication between the −X balloon subset of segment 216b and the +X balloon subset of segment 220a. A liquid inflation fluid may fill the lumen, with sufficient fluid being included to maintain the balloons at a mid-inflation state (so that the balloons are at about the middle of their range of inflation between a maximum inflation state and a nominally inflated state). Another similarly liquid-filled lumen may provide fluid communication between the balloon subset of the +X bend orientation of segment 216b, and the −X bend subset of segment 220a. A third lumen may extend between the +Y subset of segment 216b and the −Y subset of segment 220a; and a fourth lumen between the −Y subset of segment 216b and the +Y subset of segment 220a. Opposed orientations of the subsets of segments 216a and 220b may be similarly in fluid communication via associated lumens. When the handle of balloon drive 212 is bent along segment 220a in the −X orientation, the balloons of the drive segment along the inner curvature are compressed, inducing fluid flow to the balloons of segment 216b so as to generate expansion and a corresponding outer curvature along the +X orientation subset. Similar bending in the other orientations, and of the other segments, is coordinated by the coupling of laterally opposed subsets of the balloons of the associated catheter segment and the drive segment arrays, optionally with the associated segments being axially opposed as described above.

Articulation of balloon pump system 210 may be limited to lateral bending of the segments, or the handle may accommodate axial articulation input as well. For example, the resilient outer structure of the drive system handle (and/or an axially non-distensible, laterally flexible inner tension member within the balloon array) may be under tension so as to maintain a desired overall axially compressive load on the drive balloons. To allow axial input at the handle, a threaded connector such as a wing-nut 224 (shown at the proximal end of the handle) may be twisted to vary the axial length of the handle and hence the overall axial compression of the pump balloon array. Decreasing the pump array length by twisting the wing nut in one direction may induce flow from the pump balloons to the catheter balloon array, which may axially expand the catheter segments by resiliently and axially elongating a helical spring or frame structure of the segments. Alternatively, a wing-nut or other threaded connector may be mounted near the interface between the handle and the catheter to facilitate inputting axial length change input commands without altering lateral bend articulation inputs

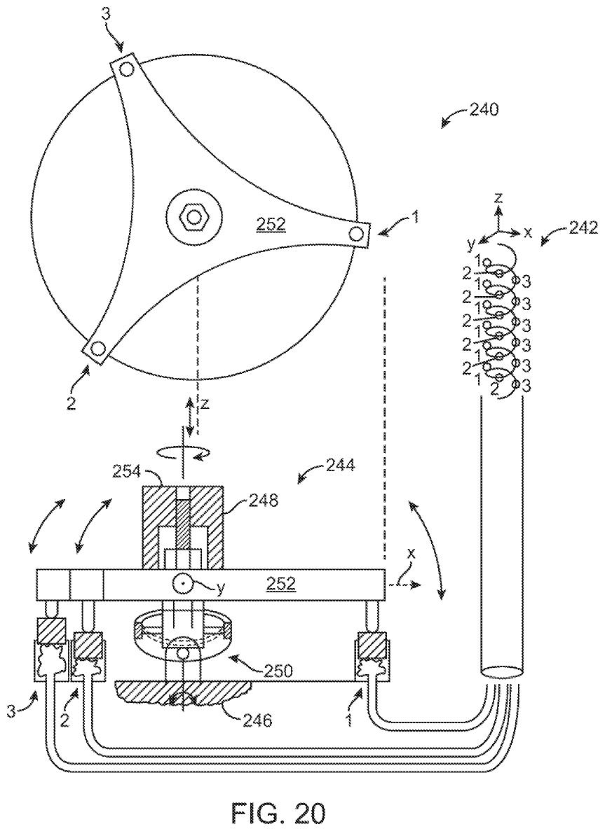

Referring now to the schematic illustration of FIG. 20, a gimbal input/balloon array system 240 includes a catheter 242 extending distally from a gimbal drive system 244. Catheter 242 includes a balloon array having, for example, a helical multi-lumen/balloon tube assembly as described above, providing first, second, and third balloon subsets 1, 2, and 3 aligned along circumferentially offset lateral bending orientations relative to an axis of the catheter. Gimbal drive system 244 includes a proximal housing 246 supporting a manual input 248 via a gimbal joint 250. The gimbal joint allows the input handle to move relative to the proximal housing in transverse rotational degrees of freedom. A gimbal plate 252 tilts with pivotal movement of the input relative to the housing, and tilting of the gimbal plate results in actuation of displacement pumps 1′, 2′, and 3′, with pistons of the pumps moving axially relative to cylinders of the pumps, compression members of the pumps pressing against balloons of the pumps, or the like. Lumens provide fluid communication between the pumps and catheter balloon subsets 1′ and 1, 2′ and 2, 3′ and 3; and fluid flows from the pumps induce lateral (X-Y) deflection of the catheter segment relative to the proximally adjacent portion of the catheter body corresponding to the lateral deflection of the input 248 relative to the housing 246. Axial articulation of the catheter segment may optionally be provided by a threaded nut 254 of input 248, where rotation of the nut induces axial (Z) displacement of gimbal plate 252 relative to the housing. Axially shortening movement of the gimbal plate toward the housing may induce inflation fluid to flow from the drive system toward the segment that increases the length of the segment.

While the exemplary embodiment have been described in some detail for clarity of understanding and by way of example, a variety of modifications, changes, and adaptations of the structures and methods described herein will be obvious to those of skill in the art. Hence, the scope of the present invention is limited solely by the claims attached hereto.

What is claimed is:

1. An articulated catheter system comprising:
an elongate catheter body having a proximal end and a distal end with an axis therebetween;
a balloon array including a first subset of balloons, the first balloon subset being axially or circumferentially distributed, or both, and offset from the axis;
a lumen in fluid communication with the first balloon subset and extending proximally; and
a first manual pump configured for coupling with the proximal end of the catheter body in fluid communication with the lumen, the first pump having a base and a handle manually movable relative to the base so as to induce a first flow of inflation fluid within the lumen such that the first subset of balloons inflates and induce a first articulation of the catheter body.

2. The articulated catheter system of claim 1, wherein the balloon array further comprises a second subset of balloons, the second balloon subset being axially distributed and circumferentially offset from the first balloon subset so that the first articulation comprises lateral bending of the catheter body along a first lateral bending axis, and inflation of the second balloon subset induces a second articulation comprising lateral bending of the catheter body along a second lateral bending axis transverse to the first bending axis.

3. The articulated catheter system of claim 1, wherein the balloon array further comprises a third subset of balloons, the third balloon subset being axially distributed and axially offset from the first balloon subset so that the first articulation comprises lateral bending of the catheter body along a first axial segment, and inflation of the third balloon subset induces a third articulation comprising lateral bending of the axis along a second axial segment axially offset from first segment.

4. The articulated catheter system of claim 1, wherein the first pump comprises a positive displacement pump, and wherein the inflation fluid comprises an inflation liquid.

5. The articulated catheter system of claim 1, further comprising a second manual pump configured for coupling with the proximal end of the catheter body in fluid communication with another lumen, the second pump having a base and a handle manually movable relative to the base so as to induce a second flow of inflation fluid within the other lumen such that the array of balloons articulates the catheter body.

6. The articulated catheter system of claim 1, wherein the balloon array includes 3 or more associated balloon subsets configured to be coupled to 3 or more associated manual pumps by three or more associated lumens so that the catheter body is configured to articulate with 3 or more degrees of freedom.

7. The articulation catheter system of claim 1, wherein the balloon array includes 6 or more subsets of balloons so that the catheter body is configured to articulate with 6 or more degrees of freedom.

8. The articulated catheter system of claim 1, wherein a first movement of the handle of the first pump relative to the base in a first input orientation induces the first articulation, and wherein a second movement of the handle of the first pump relative to the base in a second orientation induces a fourth articulation.

9. The articulated catheter system of claim 1, wherein the first pump is configured to be manually reoriented so that the first orientation of the first handle movement corresponds to an image of a first orientation of the first articulation of the catheter body, and so that the second orientation of the second handle movement corresponds to a second orientation of the second handle movement.

10. The articulated catheter system of claim 1, wherein the first pump comprises threads coupling the handle of the first pump to the base of the first pump so that rotation of the handle relative to the base induces the first flow by driving a piston of the first pump axially within a cylinder of the first pump.

11. The articulated catheter system of claim 1, wherein the first pump comprises a pump balloon in fluid communication with the first subset of balloons, movement of the handle relative to the base compressing the pump balloon so as to induce a flow on inflation fluid from the pump balloon to the subset.

12. An articulated catheter for use with a first manual pump having a base, a handle manually movable relative to the base so as to induce a first flow of inflation fluid, and a pump coupler, the catheter comprising:
an elongate catheter body having a proximal end and a distal end with an axis therebetween;
a balloon array including a first subset of balloons, the first balloon subset being axially distributed and offset from the axis;
a lumen in fluid communication with the first balloon subset and extending proximally; and
a catheter coupler adjacent the proximal end of the catheter body, the catheter coupler configured for coupling with the pump coupler so as to provide sealed fluid communication between the first pump and the lumen so that the first flow inflates the first subset of balloons and induces a first articulation of the catheter body.

13. A manual pump for use with an articulated catheter, the articulated catheter including an elongate catheter body, a balloon array including a plurality of subset of balloons, and a plurality of lumens, each lumen in fluid communication with an associated balloon subset, and a catheter coupler, the manual pump comprising:
a base;
at least one handle manually movable relative to the base so as to induce a plurality of inflation fluid flows;
a pump coupler configured for coupling with the catheter coupler so as to provide sealed fluid communication between the first pump and the lumens so that the flows inflate the subsets of balloons and each flow induces an associated articulation of the catheter body.
